(19) United States
(12) Patent Application Publication
(10) Pub. No.: US 2016/0144117 A1
(43) Pub. Date: May 26, 2016

(54) HYPODERMIC SYRINGE THAT AUTOMATICALLY EJECTS MEDICAMENT FROM A NEEDLE

(71) Applicant: Thomas Chun, Northridge, CA (US)

(72) Inventor: Thomas Chun, Northridge, CA (US)

(21) Appl. No.: 14/550,624

(22) Filed: Nov. 21, 2014

(51) Int. Cl. A61M 5/20; A61M 5/31; A61M 5/315

(52) U.S. Cl. CPC A61M 5/2033; A61M 5/31501; A61M 5/31511; A61M 5/3135

(57) ABSTRACT

An automatic injection syringe that is pre-loaded with medicament and protected from inadvertent ejection of said medicament or injection of air. A plunger assembly is first positioned with a compressed spring in between a plunger and a retainer element. The compressed spring is held in place by a resilient retainer sleeve and the plunger is inside the syringe tube. When the syringe is pushed back, the resilient retainer sleeve releases the spring which then expands to push the plunger into the reservoir of the syringe tube to eject the medicament out from a syringe needle.

### FIELD OF INVENTION

The present invention relates to a hypodermic syringe. More particularly this invention concerns a syringe preloaded with the desired amount of medicament that is ready to be injected into a patient. After the needle of the syringe is forced into the patient, a plunger automatically pushes the medicament into the patient through the needle.

### DESCRIPTION OF PRIOR ART

A hypodermic syringe is described as having a generally cylindrical syringe tube centered on a longitudinal axis and having a hollow needle with an open tip attached to the front end. A plunger is slidable inside the tube and define the liquid compartment into which the medicament is placed. When the plunger is moved forward toward the needle, the medicament is pushed though the needle and out the open tip of the needle so that the medicament is injected into the patient.

Hypodermic syringes are primarily used to effect subcutaneous injections. It is well known in the art that many patients are injected with the same dosage of the same medicament. Thus, in preparation, medical professionals often pre-load multiple syringes with the same dosage of the same medicament. When the syringes are pre-loaded, the plunger is left in the retracted position ready to be pushed toward the needle so as to inject the medicament. However, a common problem encountered is that the plunger can be moved forward or backward inadvertently before it is injected into a patient. As this occurs, some of the medicament is pushed out or air is pulled into the tube of the syringe. Either of these scenarios is highly undesirable because it results in the medicament to be contaminated with air or the dosage of the medicament in the syringe to be reduced. Whether air enters the tube of the syringe or some of the medicament is pushed out of the syringe, such pre-loaded syringe becomes unusable and must often be discarded. This problem is primarily caused by the plunger that is unprotected and susceptible to inadvertent movement forward or backward relative to the tube of the syringe.

Accordingly, there is a need for a syringe with a plunger that is protected or encased so that it does not move forward or backward inadvertently until the medicament is ready to be injected into the patient.

### SUMMARY OF THE INVENTION

Accordingly, the present invention has been made in view of the above-mentioned disadvantages occurring in the prior art. The present invention is an automatic injection syringe that is pre-loaded with the desired dosage of a medicament and can be stored for extended periods of time without fear that the plunger will inadvertently be pushed or pulled rendering the syringe useless.

It is therefore the primary object of the present invention to provide a syringe with a plunger that is encapsulated or encased so that it may not be inadvertently pushed or pulled.

Another object of the present invention is to provide a syringe that can be pre-loaded with the desired amount of medicament.

Yet another object of the present invention is to provide a syringe that automatically injects the medicament into the patient once the needle is forced into the patient.

A still further object of the present invention is to provide a syringe with components that can be varied so as to control the rate by which the medicament injected into the patient.

A yet further object of the present invention is to provide a syringe that is cost effective to manufacture.

The above objects and other features and advantages of the present invention, as well as the structure and operation of various embodiments of the present invention described in detail with reference to the accompanying drawings.

### DESCRIPTION OF THE DRAWINGS

The accompanying drawings which are incorporated by reference herein and form part of the specification, illustrate various embodiments of the present invention and, together with the description, further serve to explain the principles of the invention and to enable a person skilled in the pertinent art to make and use the invention. In the drawings, like reference lumbers indicate identical or functional similar elements. A more complete appreciation of the invention and many of the attendant advantages thereof will be readily obtained as the same becomes better understood by reference to the following detailed description when considered in connection with the accompanying drawings, wherein:

FIG. 1 is a perspective view of the automatic injection syringe of the present invention in its assembled state as it would be assembled before use on a patient.

FIG. 2 is an exploded view of the automatic injection syringe of the present invention.

FIG. 3 is a perspective view of the tube of the present invention.

FIG. 4 is a perspective view of the shell of the present invention.

FIG. 5 is a perspective view of the shell cap of the present invention.

FIG. 6 is a perspective view of the plunger of the present invention.

FIGS. 7a and 7b are a perspective views of the retainer of the present invention.

FIGS. 8a and 8b are a perspective views of the retainer sleeve of the present invention.

FIG. 9 is a perspective view of the plunger assembly of the present invention.

FIG. 10 is an exploded view of the plunger assembly of the present invention.

FIG. 11 is a cross-sectional view of the automatic injection syringe of the present invention in its assembled state as it would be before use on a patient.

FIG. 12 is a cross-sectional view of the automatic injection syringe of the present invention in its assembled state as it would be after use on a patient.

### DETAILED DESCRIPTION OF THE PREFERRED EMBODIMENTS

Reference will now be made to the drawings in which various elements of the present invention will be given numerical designations and in which the invention will be discussed so as to enable one skilled in the art and make use the invention.

The present invention comprises an automatic injection syringe 100 comprising a syringe tube 10, a shell 20, a shell cap 30, a needle 40, a sleeve 45, a needle cap 43, and a plunger assembly 90 which comprises a plunger 50, a spring 60, a retainer 70, and a retainer sleeve 80. It is well known that in the treatment of patients, medical professionals often inject the same dosage of the same medicament. Thus, as a time saving exercise, it is customary for medical professionals to pre-load multiple syringes with the same dosage of the same medicament ready for use. However, conventional syringes comprise a plunger that is exposed and can be manually displaced inadvertently so as to release some of the medicament and render the pre-loaded syringe useless. The automatic injection syringe 100 of the present invention allows a syringe to be pre-loaded with medicament and encapsulates the plunger so as to eliminate the risk that it will be inadvertently displaced. Then when needed, the medical professional can inject the medicament into a patient without pressing or even touching the plunger.

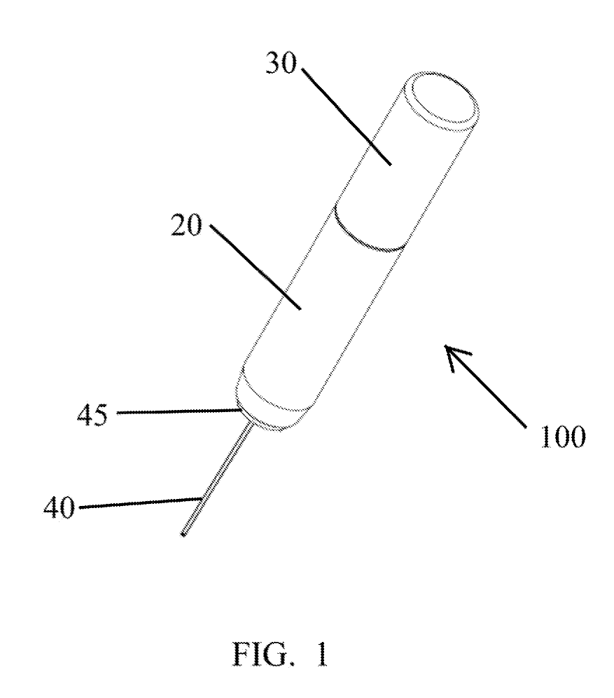

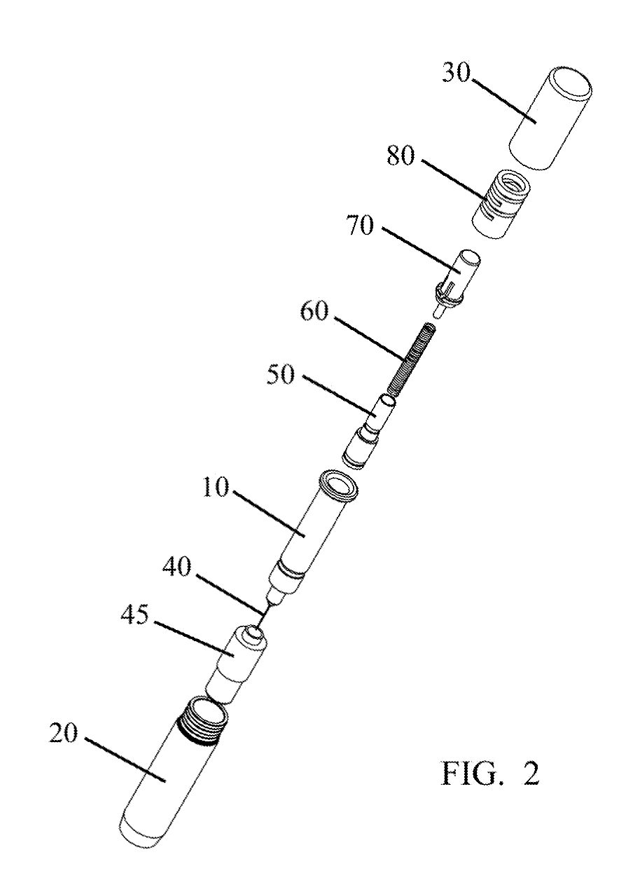

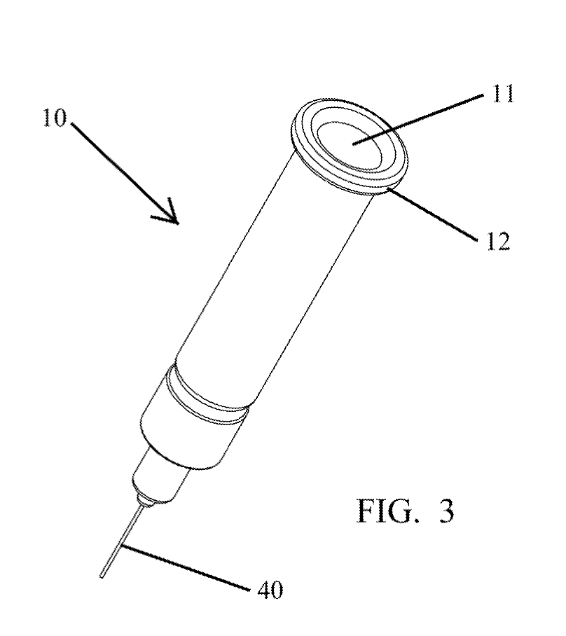

FIG. 1 shows a perspective view of the automatic injection syringe 100 of the present invention in its assembled state as it would be used. FIG. 2 shows an exploded view of the automatic injection syringe 100 of the present invention to depict in greater detail the various components that comprise the automatic injection syringe 100. The main component of the present invention is the tube 10 having an opening 11 in one end and the needle 40 with the sleeve 45 attached to the opposing end. The needle 40 is hollow with an open tip and in fluid communication with the interior of the tube 10. Accordingly, any fluid or medicament inside the tube 10 can be forced or pushed to be discharged through the needle 40 out the open tip. An important element of the tube 10 is an annular flange 12 that extends outwardly around the opening 11, as shown in FIG. 3.

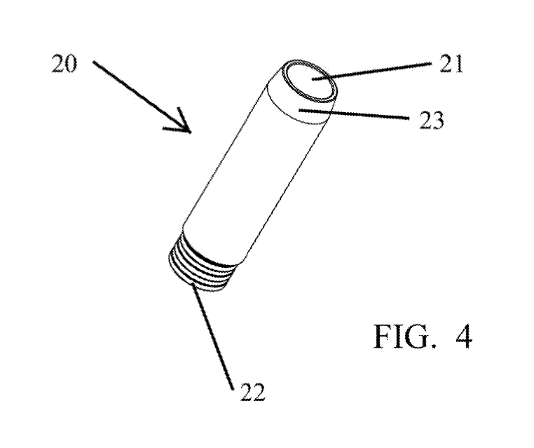

FIG. 4 shows a perspective view of the shell 20 that is tubular in shape and having a front opening 21 and a rear opening 22. However, the rear opening 22 is larger in diameter than the front opening 21. The preferred design of the shell 20 includes a taper 23 towards the front to create the smaller front opening 21, as shown in FIG. 4.

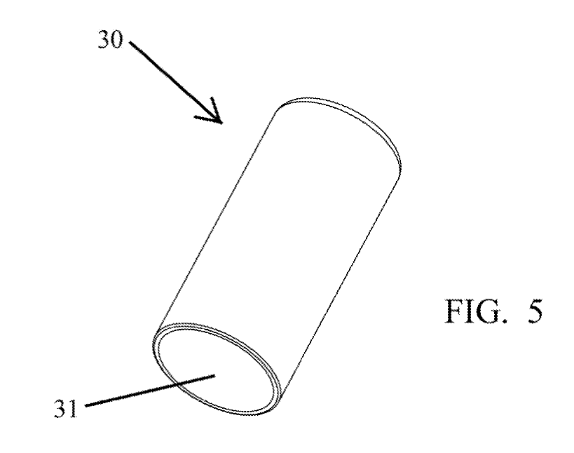

FIG. 5 shows a perspective view of the shell cap 30 that is also tubular in shape and having an opening 31 in one end while the other end is closed. The design configuration of the shell 20 and the shell cap 30 is such that the shell cap 30 can be attached to the rear opening 22 of the shell 20 so as to cover or cap it. Furthermore, the diameter of the rear opening 22 of the shell 20 is large enough for the tube 10 to be inserted therethrough but small enough for the annular flange 12 not to pass through. Thus, the tube 10 can be inserted into the shell 20 until the annular flange 12 rests against the rear opening 22. Additionally, the length of the shell 20 is smaller than the length of the tube 10 such that when the tube 10 is inserted into the shell 20 until the annular flange 12 rests against the rear opening 22, a portion of the sleeve 45 is exposed and protrudes past the front opening 21 of the shell 20, as shown in FIG. 1.

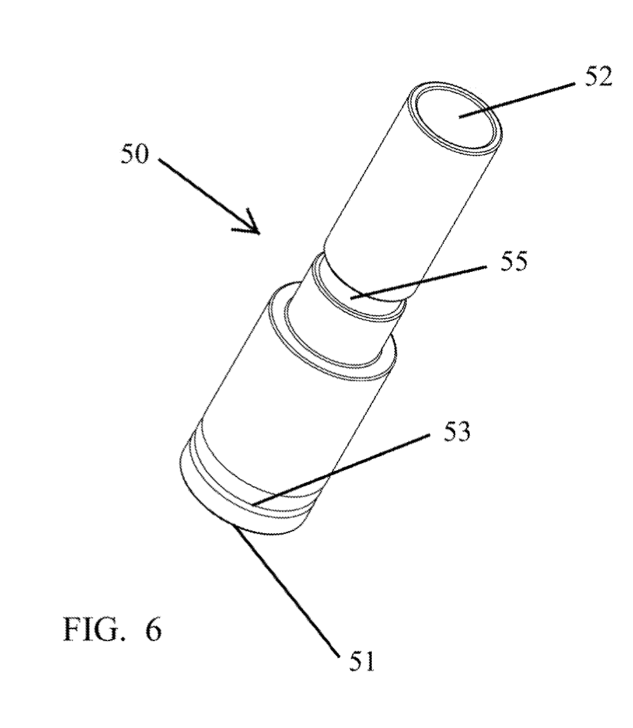

FIG. 6 shows a perspective view of the plunger 50 that is tubular in shape having a closed, end 51 and an opening 52 in the opposing end. In addition, an o-ring 54 is used around the plunger 50 to provide a radial seal. Finally, the plunger 50 has an annular groove 55 as shown in FIG. 6. The spring 60 is inserted into the plunger 50 through the opening 52.

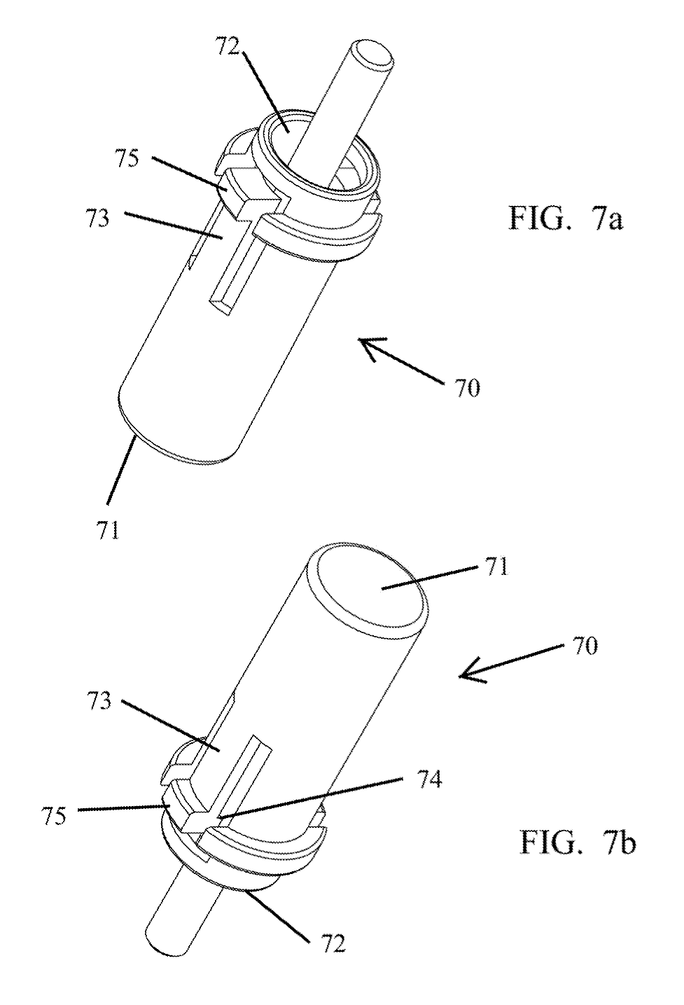

FIGS. 7a and 7b shows a perspective view of the retainer 70 that is tubular in shape having a closed end 71 and an opening 72 in the opposing end. Additionally, the retainer 70 has two flexing elements 73 which extend along the sidewall of the retainer 70 and can flex radially inward and outward. Each flexing element 73 has an inner peg 74 that protrudes radially inward and an outer peg 75 that protrudes radially outward, as shown in FIGS. 7a and 7b.

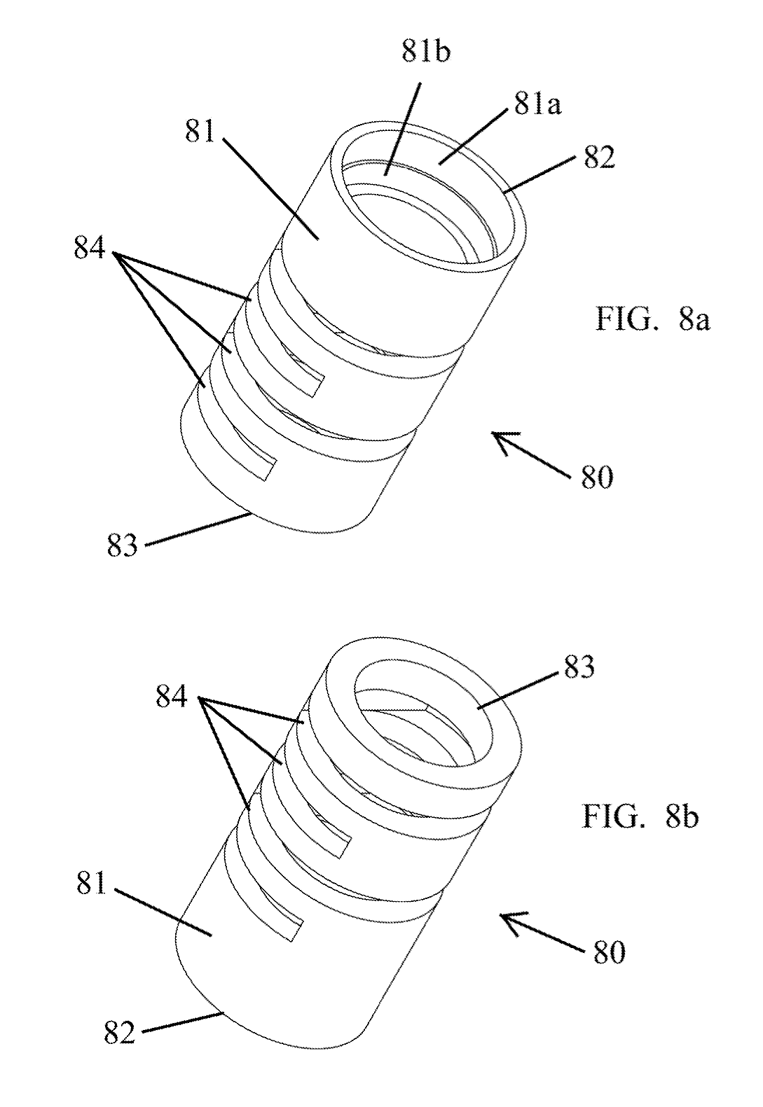

FIGS. 8a and 8b shows a perspective view of the retainer sleeve 80 that is tubular in shape with a base 81 and a base opening 82 in one end and a top opening 83 in the opposing end. The base 81 has a large section 81a and a small section 81b such that the inner diameter of the large section 81a of the base 81 is larger than the inner diameter of the small section 81b of the base, as shown in FIG. 8a. Above the base 81, the retainer sleeve 80 has various groves 84 that are configured to allow the retainer sleeve 80 to be compressed longitudinally.

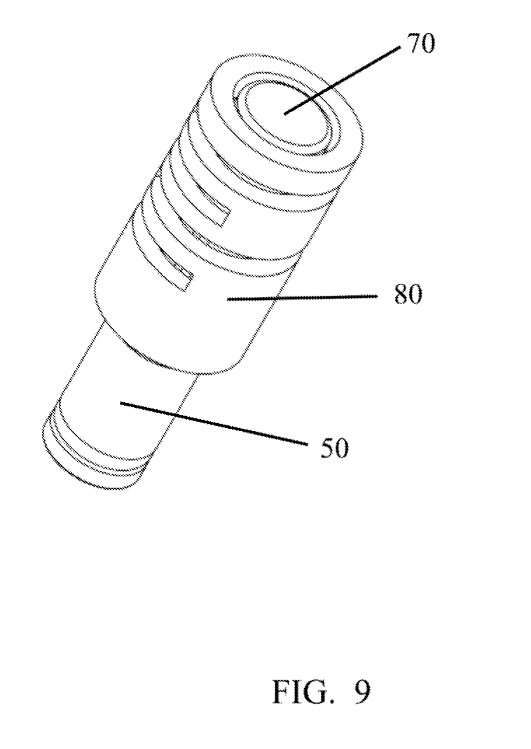

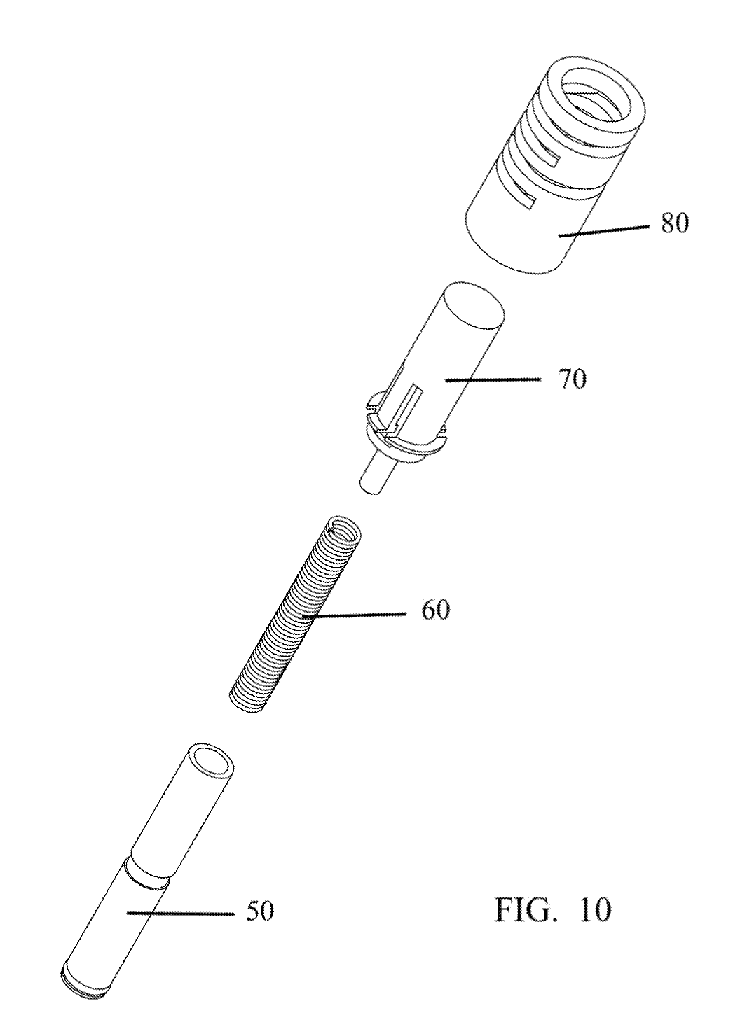

FIGS. 9 and 10 show a perspective and an exploded view, respectively, of the plunger assembly 90. The plunger assembly 90 is the assembly of the plunger 50, the spring 60, the retainer 70, and the retainer sleeve 80. The plunger assembly 90 is assembled by first inserting the spring 60 into the plunger 50 through the opening 52 until it rests against the closed end 51. Then the retainer 70 is placed over the spring 60 such that the spring 60 is inserted through the opening 72. Then the retainer 70 is pushed toward the plunger 50 so as to compress the spring 60 until the inner pegs 74 on the retainer 70 are aligned with the annular groove 55 on the plunger 50. Then the retainer sleeve 80 is placed over the retainer 70 such that the closed end 71 of the retainer 70 is passed through the base opening 82 until the outer pegs 75 are within the large section 81a of the base 81 of the retainer sleeve 80. Then, the flexing elements 73 of the retainer 70 are flexed inward so that the inner pegs 74 are inserted into the annular groove 55. Finally, the retainer sleeve 80 is pushed further toward the plunger 50 until the outer pegs 75 are pushed into the small section 81h of the base 81 of the retainer sleeve 80 so that the inner pegs 74 are held within the groove 55. Accordingly, the plunger assembly 90 is maintained assembled by the inward flexing of the flexing elements 73 such that the inner pegs 74 are retained within the groove 55, thus, mechanically locking the plunger assembly 90, as shown in FIG. 9.

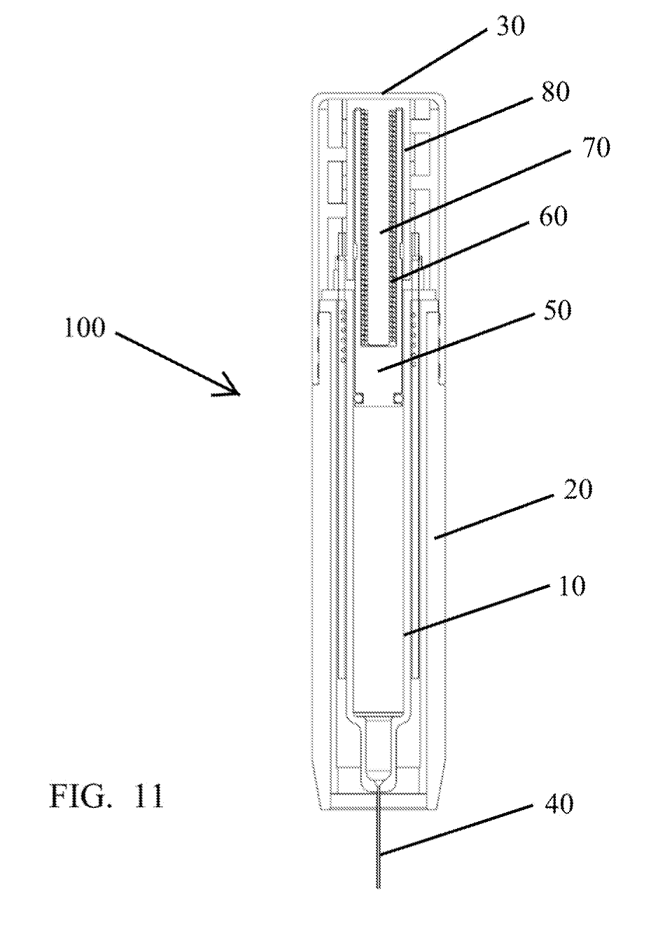

Once the plunger assembly 90 is assembled, it is coupled with the tube 10 through the opening 11 so that the plunger 50 is inside the tube 10 and the base 81 of the retainer sleeve 80 rests on the annular flange 12 of the tube 10. With the plunger assembly 90 in place, the tube 10 is inserted into the shell 20 through the rear opening 22 until the annular flange 12 rests against the rear opening 22 and a portion of the sleeve 45 is exposed and protrudes past the front opening 21 of the shell 20. Finally, the shell cap 30 is attached to the rear opening 22 of the shell 20 so as to cover or encapsulate the entire tube 10 and plunger assembly 90 within the shell 20 and the shell cap 30. Once the shell cap 30 is attached to the shell 20, the automatic injection syringe 100 of the present invention is ready for use, as shown in FIG. 11. However, when immediate use is not necessary or when storage of the automatic injection syringe 100 is required, the needle cap 43 is used to cover the needle 40 so that it does not inadvertently poke or puncture somebody.

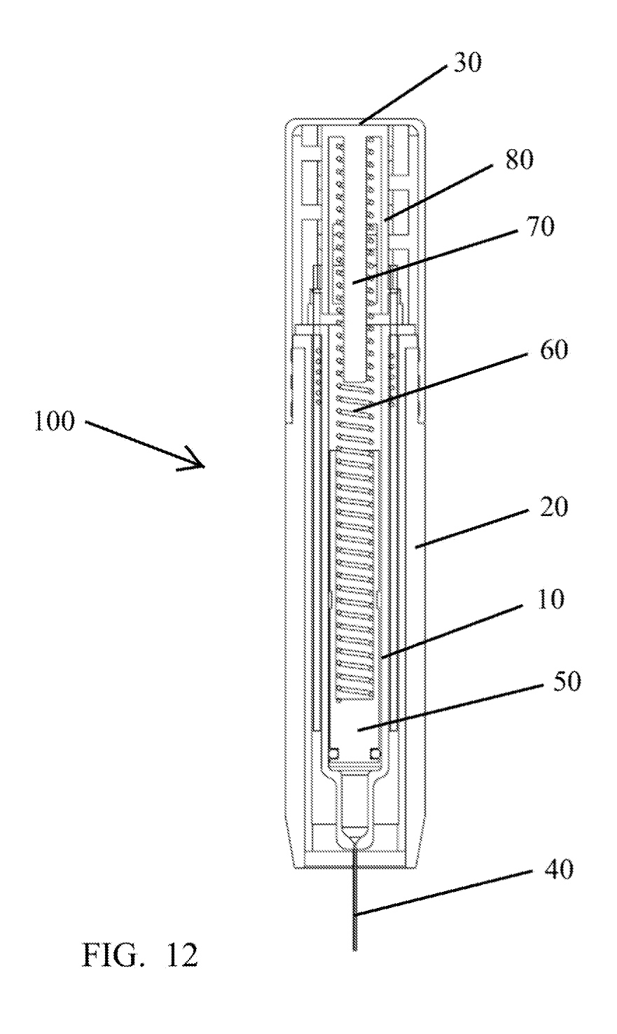

When the automatic injection syringe 100 of the present invention is ready for use to inject medicament into a patient, the needle cap 43 is removed and the needle 40 is forced into the patient in such a manner and to such extent as to press against, the portion of the sleeve 45 that is exposed and protrudes past the front opening 21 of the shell 20. In doing this, the tube 10 is pushed back so as to longitudinally compress the retainer sleeve 80. As the retainer sleeve 80 is pushed back longitudinally, the large section 81a of the base 81 is aligned with the outer pegs 75 so as to allow the flexing elements 73 to displace to its natural position, thus, releasing, the inner pegs 74 that are held within the groove 55. This allows the spring 60 to extend to its natural uncompressed length, thus, pushing the plunger 50 toward the needle 40, as shown in FIG. 12. As the force of the spring 60 pushes the plunger 50, the medicament is forced to discharge through the needle 40 and into the patient.

It is well known in the field of art that different medicaments have to be injected with different injection rates. Thus, the automatic injection syringe 100 of the present invention can be configured with a spring 60 having a compressibility ration that corresponds the desired injection rate. This way, when the spring 60 is released, the stiffness of the spring 60 will allow the plunger 50 to be pushed forward at the desired injection rate. For example, the stiffer the spring 60, the greater the force with which the plunger 50 will be pushed, thus, resulting in a greater injection rate. Similarly, the less stiff the spring 60, the lower the force with which the plunger 50 will be pushed, thus, resulting in a lower injection rate.

Therefore, the automatic injection syringe 100 of the present invention is superior in design and functionality than any existing prior art. It is understood that the described embodiments of the present invention are illustrative only, and that the modifications thereof may occur to those skilled in the art. Accordingly, this invention is not to be regarded as limited to the embodiments disclosed, but to be limited only as defined, by the appended claims herein.

What is claimed is:

1. An automatic infection syringe comprising:
a syringe tube having a front end and a rear end with a flange wherein said syringe tube defines a reservoir within which a medicament is contained;
a needle attached to said front end of said syringe tube and in fluid communication with said reservoir;
a sleeve attached to said front end of said syringe tube;
a shell having a rear end through which said syringe tube is inserted until said flange abuts said rear end such that said needle and said sleeve extend out from a front end of said shell;
a plunger assembly comprising a plunger, a spring, a retainer, and a retainer sleeve;
wherein said spring having a first end that engages said plunger and a second end that engages said retainer;
wherein, said retainer sleeve is resilient so as to releasably engage together said plunger, said spring, and said retainer such that said spring is compressed between said plunger and said retainer;
wherein said plunger assembly is coupled with said syringe tube such that said plunger is inserted through said rear end of said syringe tube until said retainer sleeve abuts said flange of said syringe tube;
a cap that is attached to said rear end of said shell and that has a cavity within which said plunger assembly is encapsulated; and
wherein said retainer sleeve disengages said plunger, said spring, and said retainer when said sleeve is pushed into said shell such that said spring expands so as to push said plunger into said reservoir of said syringe tube such that said medicament is pushed through said needle for ejection therefrom.

2. The automatic injection syringe of claim 1 wherein said spring has a stiffness that is proportional to a rate of ejection of said medicament.

3. The automatic injection syringe of claim 1 wherein said plunger further comprises an o-ring that creates a radial seal against said syringe tube such that said medicament does not leak out of said reservoir.

4. The automatic injection syringe of claim 1 wherein said shell and said syringe tube are made of clear material so as to maintain visibility of said medicament in said reservoir.

5. The automatic injection syringe of claim 1 wherein said retainer sleeve has a stiffness that is proportional to the amount of force required to push said sleeve into said shell.

6. The automatic injection syringe of claim 1 wherein said plunger assembly further comprising:
said retainer having a plurality of flexing elements having an inner peg;
wherein said inner peg engages a groove in said plunger when said spring is compressed and said flexing elements are flexed radially inward; and
wherein said retainer sleeve disengages said plunger, said spring, and said retainer when said flexing elements are returned to their natural unflexed position.

7. The automatic injection syringe of claim 6 wherein said retainer sleeve further comprising:
a first inner diameter that is sized to flex radially inward said flexing elements of said retainer; and
a second inner diameter that is sized to allow said flexing elements of said retainer return to their natural unflexed position.

8. An automatic injection syringe comprising:
a syringe tube having a reservoir within which a medicament is contained;
a needle attached to a front end of said syringe tube and in fluid communication with said reservoir;
a shell into which said syringe tube is inserted until said needle extends out from said front end;
a plunger assembly comprising a plunger, a spring, a retainer, and retainer sleeve;
wherein said spring having a first end that engages said plunger and a second end that engages said retainer;
wherein said retainer sleeve is resilient so as to releasably engage together said plunger, said spring, and said retainer such that said spring is compressed between said plunger and said retainer;
wherein said plunger of said plunger assembly is inserted into said syringe tube;
a cap that is attached to said shell and that has a cavity within which said plunger assembly is encapsulated; and
whereby pushing said syringe tube towards a rear end of said shell causes said retainer sleeve to compress so as to disengage said plunger, said spring, and said retainer such that said spring expands to push said plunger into said reservoir of said syringe tube such that said medicament is pushed through said needle for ejection therefrom.

9. The automatic injection syringe of claim 8 wherein said spring has a stiffness that is proportional to a rate of ejection of said medicament.

10. The automatic injection syringe of claim 8 wherein said plunger further comprises an o-ring that creates a radial seal against said syringe tube such that said medicament does not leak out of said reservoir.

11. The automatic injection syringe of claim 8 wherein said shell and said syringe tube are made of clear material so as to maintain visibility of said medicament in said reservoir.

12. The automatic injection syringe of claim 8 wherein said retainer sleeve has a stiffness that is proportional to the amount of force required to push said sleeve into said shell.

13. The automatic injection syringe of claim 8 wherein said plunger assembly further comprising:
said retainer having a plurality of flexing elements having an inner peg;
wherein said inner peg engages a groove in said plunger when said spring is compressed and said flexing elements are flexed radially inward; and
wherein said retainer sleeve disengages said plunger, said spring, and said retainer when said flexing elements are returned to their natural unflexed position.

14. The automatic injection syringe of claim 13 wherein said retainer sleeve further comprising:
a first inner diameter that is sized to flex radially inward said flexing elements of said retainer; and
a second inner diameter that is sized to allow said flexing elements of said retainer return to their natural unflexed position.

15. An automatic injection syringe comprising:
a syringe tube having a reservoir within which a medicament is contained;
a needle attached to a front end of said syringe tube and in fluid communication with said reservoir;
a plunger assembly comprising a plunger, a spring, a retainer, and retainer sleeve;
wherein said spring having a first end that engages said plunger and a second end that engages said retainer;
wherein said retainer sleeve is resilient so as to releasably engage together said plunger, said spring, and said retainer such that said spring is compressed between said plunger and said retainer;
wherein said plunger of said plunger assembly is inserted into a rear end of said syringe tube;
a cap having a cavity within which said plunger assembly is encapsulated; and
means for disengaging said plunger, said spring, and said retainer such that said spring expands to push said plunger into said reservoir of said syringe tube such that said medicament is pushed through said needle for ejection therefrom.

16. The automatic injection syringe of claim 15 wherein said spring has a stiffness that is proportional to a rate of ejection of said medicament.

17. The automatic injection syringe of claim 15 wherein said plunger further comprises an o-ring that creates a radial seal against said syringe tube such that said medicament does not leak out of said reservoir.

18. The automatic injection syringe of claim 15 wherein said syringe tube are made of clear material so as to maintain visibility of said medicament in said reservoir.

19. The automatic injection syringe of claim 15 wherein said retainer sleeve has a stiffness that is proportional to the amount of force required to push said sleeve into said shell.

20. The automatic injection syringe of claim 15 wherein said plunger assembly further comprising:
said retainer having a plurality of flexing elements having an inner peg;
wherein said inner peg engages a groove in said plunger when said spring is compressed and said flexing elements are flexed radially inward; and
wherein said retainer sleeve disengages said plunger, said spring, and said retainer when said flexing elements are returned to their natural unflexed position.

21. The automatic injection syringe of claim 20 wherein said retainer sleeve further comprising:
a first inner diameter that is sized to flex radially inward said flexing elements of said retainer; and
a second inner diameter that is sized to allow said flexing elements of said retainer return to their natural unflexed position.
